(19) United States
(12) Patent Application Publication
(10) Pub. No.: US 2011/0104719 A1
(43) Pub. Date: May 5, 2011

(54) Methylarginine detection in cells and tissues

(76) Inventor: John M. Aletta, Williamsville, NY (US)

(21) Appl. No.: 12/925,961

(22) Filed: Nov. 2, 2010

Related U.S. Application Data
(60) Provisional application No. 61/257,220, filed on Nov. 2, 2009.

(51) Int. Cl. G01N 33/574; G01N 33/567

(52) U.S. Cl. 435/7.23; 435/7.21

(57) ABSTRACT

The present invention provides a method for identifying a biological sample as cancerous or normal by comparing the methylation status of arginine proteins in a test sample with the normal sample. A different level of binding or pattern of binding of antibodies which are specific for arginine containing proteins in their methylated state indicates an abnormal condition.

### CROSS-REFERENCE TO RELATED APPLICATIONS

This application claims priority to U.S. Provisional application No. 61/257,220, filed on Nov. 2, 2009, the disclosure of which is incorporated herein by reference.

### FIELD OF THE INVENTION

The present invention relates to the field of methylation status of arginine containing protein. In particular, this invention provides a method for detection of cancer and other disorders based on the level or pattern of methylarginine proteins.

### BACKGROUND OF THE INVENTION

Protein methylation is considered to play an important role in cellular functions such as signaling. In eukaryotic cells, proteins are methylated on carboxyl groups or on the side chain nitrogen of the amino acids lysine, arginine or histidine.

N-methylation, such as that occurring on arginine residues in proteins, has generally been regarded as a constitutive and irreversible post-translational modification. However, there may be exceptions to this view. For example, NGF has been reported to dramatically alter the pattern of protein methylation observed in PC12 cells after metabolic radiolabeling of proteins in intact cells, and by in vitro labeling of proteins in cell extracts.

While many different biomarkers for the presence of cancer or determining its severity have been proposed, there continues to be a need for identifying reliable and additional markers. While protein methylation is recognized to be important in cell signaling mechanisms, its precise role in cell growth and differentiation or neoplastic diseases remains to be elucidated.

### SUMMARY OF THE INVENTION

The present invention provides a method for diagnosis of cancer or determining the stage of cancer based upon an altered status of arginine methylation of proteins in a tissue. The altered status can be in the form of an altered level of methylation or an altered pattern of the presence of methylarginine-containing proteins in cells. This method can also be used for detecting the presence of or severity of other diseases in which the methylation level of arginine containing proteins is altered or the sub-cellular localization of such proteins is altered. This method also provides kits for the detection of methylation status of arginine containing proteins.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Colon cancer cells exhibit a markedly divergent pattern of methylarginine protein staining when compared to normal colon tissue. The typical staining pattern observed in normal colon tissue appears punctuate and is illustrated in the left panel. Tumor tissue exhibits a variation in the sub-cellular staining pattern. A diffuse, cytoplasmic pattern with little or no nuclear staining is illustrated in the right panel. Both tumor tissue and adjacent normal tissue are removed from the same patient.

FIG. 2. Nuclear versus cytoplasmic staining pattern is reversed in normal versus neoplastic thyroid tissue. Normal human thyroid tissue sections (upper slide section) stained with the methylarginine-specific antibody exhibit intense reaction with nuclear methyl-proteins and a relatively much fainter signal in the cytoplasmic compartment of the cells. In the case of thyroid papillary carcinoma (lower slide section), the staining pattern is reversed. Methylarginine staining is predominantly cytoplasmic with little to no reaction with nuclear proteins. Although this dichotomy has been observed in a much smaller series of patients than the other cancers analyzed, the results are consistent across all patients.

FIG. 3. Methylarginine protein expression in pancreatic endocrine tumors (PET). Representative images taken from a series of 136 tumor samples derived from pancreatic endocrine tumors at low (20×) and high (63×, inset) magnification. A, D, G samples show a predominant cytoplasmic staining, B, E, H a mixed cytoplasmic and nuclear staining, while C and I show low to undetectable staining or low to undetectable with juxtanuclear golgi-like localization of the residual signal (F). All the samples were treated under identical experimental conditions, thus allowing the comparison of the relative signal intensities and localizations.

FIG. 4. Additional examples of neoplastic colon cells stained for methylarginine. Three types of staining cay be observed; staining may be “punctate” in a peri-nuclear golgi-like pattern which is also typical of normal tissue, predominantly “cytoplasmic” or a “mixed” staining pattern showing elements of both staining types.

FIG. 5. Distinctive patterns and levels of methylarginine protein biomarker are detected in colon, gastric and breast cancers. Tumor samples stained with the methylarginine-specific antibody, anti-mRG are illustrated: Example of a colon cancer showing cytoplasmic methylarginine expression (upper panel) and of a colon cancer with perinuclear/Golgi-like signal (lower panel); Examples of gastric and breast cancers with high methylarginine cytoplasmic expression are shown in the upper panel, and cases with a faint signal in the lower panels. The inset (63×) shown in the lower right image (breast cancer) displays a mitotic figure in which the methylarginine signal is clearly higher than that of the surrounding cells. (20×).

### DETAILED DESCRIPTION OF THE PREFERRED EMBODIMENTS

The present invention is based on the finding that cancerous tissues were found to exhibit a different level of methylation of arginine containing proteins and/or pattern of localization of methylated form of such proteins. Thus, this invention provides a method for detection of methylation status of arginine containing proteins in cells and tissues and based on the presence or subcellular localization of the proteins, determining the presence or absence of neoplasia or the severity of the neoplastic disorder. The methylarginine containing proteins are detected by using affinity molecules that specifically bind to the proteins when one or more arginines on the protein is/are methylated, but do not specifically bind when the arginines are not methylated.

In one embodiment, the affinity molecule is an antibody that specifically binds to a peptide or protein when one or more arginines in the protein or peptide are methylated but does not specifically bind to the protein or peptide when the one or more arginines are not methylated. The epitope that the antibody binds to may be an epitope comprising at least 5 amino acids, and may comprise one or more GRG motifs. A suitable antibody is described in U.S. Pat. No. 6,699,673, the disclosure of which is incorporated herein by reference. Such antibodies may recognize a wide variety of proteins when the arginine in these proteins is methylated. Alternatively, antibodies may be developed that recognize particular proteins in their arginine-methylated form. One example of such an antibody is an antibody that recognizes the methylated form of the alpha sub-type of the human estrogen receptor (ERα/NR3A1) (Le Romacer et al., Molec. Cell 31: 212-221, 2008). Similarly antibodies which recognize other arginine containing proteins in their methylated state can be used.

Thus, in one embodiment, the invention provides a method for distinguishing a normal biological sample from a cancerous biological sample comprising the steps of comparing the level of binding or pattern of binding of antibodies specific to methylarginine proteins in the normal biological sample and a suspected cancerous biological sample, and identifying the suspected cancerous biological sample as cancerous if the level or pattern of binding is different from the level or pattern in the normal sample.

The present method can also be used to detect the presence of, or severity of diseased conditions other than cancer. Such conditions include, but are not limited to, scleroderma, systemic lupus erythematosus, multiple sclerosis, Fragile X/FXTAS syndrome, Rett syndrome, spinal muscular atrophy, HIV infections, cardiovascular diseases. The proteins whose methylation level or pattern is relevant for this invention include, but are not limited to, fibrillarin, Smith protein, coilin, heterogeneous nuclear ribonucleoproteins (hnRNPs), myelin basic protein, fragile X mental retardation protein (FMRP), methyl-CpG binding protein 2 (MeCP2), Tat, hepatitis delta antigen and the like. Not all proteins, nor even proteins that contain arginine residues, are capable of undergoing methylation at arginine residues. The majority of proteins that are modified by post-translational methylation of arginine tend to exhibit glycine-arginine rich (GAR) domains of amino acids that appear to be preferred sites of protein arginine methyltransferase (PRMT) activity. The diversity of methylarginine-containing proteins is widespread and includes examples of single arginine residues that occur in otherwise nondescript amino acid domains and which are methylated by PRMTs. An example of this is the protein, DNA polymerase β (E1 Andaloussi et al., Molec. Cell 22: 51-62, 2006; FASEB J. 21: 26-34, 2007). In one embodiment, antibodies raised against GAR or single arginine methylated proteins can serve suitably as affinity molecules for the detection of the methylated forms of the respective proteins.

The methods of the present invention may be employed with any biological sample in which the status of protein methylation is to be determined. Biological samples taken from human subjects for use in the methods disclosed herein include biological fluids such as serum, blood plasma, blood borne cells, fine needle aspirate, ductal lavage, bone marrow sample or ascites fluid, as well as, tissue samples such as from normal tissue or abnormal tissue (e.g., benign or malignant tumor tissue). The tissue samples may be obtained via biopsy or autopsy or the like. In one embodiment, circulating cells (such as white blood cells) can also be used to determine the status of protein methylation. The white blood cells or other cells obtained via biopsy or autopsy may be used fresh, or may be cultured before further processing.

In one embodiment, the invention provides a method for detecting whether a biological sample contains arginine-methylated proteins—or in other words, whether the arginines in proteins are methylated. The steps of the process comprise obtaining a biological sample suspected of containing arginine-methylated proteins; if necessary, processing the biological sample for detection of affinity molecule labeling; contacting the biological sample with an affinity molecule (such as an antibody) that specifically binds to arginine-methylated proteins; and detecting the presence of the bound affinity molecules, wherein the presence of the bound affinity molecules indicates the presence of arginine-methylated proteins in the sample. In one embodiment, the affinity molecule is an antibody which specifically binds to GRG motif in proteins when the arginine in the motif is methylated (such as the antibody described in U.S. Pat. No. 6,699,673). The detection of bound antibodies can be carried out by using routine methods such as labeled secondary affinity molecules (i.e. secondary antibodies). These secondary antibodies can be in the form of fluorophore-tagged, enzyme-linked (e.g. horseradish peroxidase, alkaline phosphatase) or radioisotope-tagged antibodies that are used to report the presence of the bound primary methylarginine-specific antibody by means of a fluorescence detector, colored reaction product deposition or ionizing radiation detection, respectively.

The tissue or cells can be stained fresh or processed by any suitable means such as paraffin embedding or cryostat sectioning. In one embodiment, the tissue is fixed by routine fixation techniques (such as in formalin) and then embedded in a suitable embedding medium (such as paraffin). After sectioning and labeling with methylarginine-specific antibodies, the sample is ready for review and assessment by a suitable individual (such as a pathologist or a trained lab technician).

The present invention can be used to determine the methylation status of proteins in a wide variety of applications—including, but not limited to, cancer, neurodevelopmental diseases, autoimmune, viral, and cardiovascular diseases. It was observed that the arginine methylation status in tumor tissue was different from the arginine methylation status of normal tissue. As an example, three different types of human tumor specimens were evaluated. Based on the observations in the present invention, a determination of an altered status of arginine methylation in a biological sample can be made based upon the amount of methylation or the pattern of methylation. Distinctive staining patterns of methylarginine protein distribution in the tumor tissue can be read by an individual (such as a pathologist) to place the tumor in specific categories indicative of disease status, prognosis and/or personalized treatment options likely to be of greatest benefit to the patient. To rule out specious staining artifacts, a control antibody can be compared with the methyl-specific staining using serial sections from the same biopsy material. The control antibody is raised against a peptide identical to the antigen used to generate the methylarginine-specific reagent except for the absence of any methyl groups in the control peptide antigen. The control or normal sample for comparison can be obtained from the same individual, or from a normal individual who is free of the disease. When a suspected individual's tissue staining is compared to a normal individual's staining, the normal sample can be run in parallel or the normal staining pattern can be obtained from archival data.

Variations in methylarginine detection that are characteristic of neoplasm differ from normal in either the level or the location of the methylarginine-containing proteins. A clinical pathologist, a trained technician, or other such personnel, can easily determine alterations in the cellular location of detected methylarginine proteins by the means described here. In those cases in which the level or intensity of the methylarginine protein detected by affinity molecules is reduced in neoplasms as described in the present examples, three independent reviewers could assign relative quantitative grades of “0, negative, ±, barely detectable, +, positive or ++, strongly positive”. Additional quantitative assessments of the expression of methylarginine-containing proteins such as whole slide imaging and analysis can also be employed with the embodiments described. Online virtual imaging, digitization of pathology slides, scoring and high throughput image analysis can also be used to provide research/diagnostic tools to facilitate tumor analysis and advanced tissue diagnostics/discovery for clinical use of methylarginine detection. Objective quantitative scoring of digital images derived from tissue section immunohistochemical staining collated with other clinical and pathological patient metadata can be made readily available online for review and discussion by research and clinical teams. Platforms are currently available from virtual pathology laboratories that enable the online viewing and manual scoring of virtual slides. Virtual slides are digitized images of glass slides containing tissue samples. Glass slides are scanned to produce a high quality digital image at high resolution for accurate diagnostic work that may be shared online. Such technology may be used to support image analysis of virtual slides by developing specific algorithms to evaluate not only staining levels (intensity), but also patterns obtained from the application of methylarginine-detection strategies based on this invention.

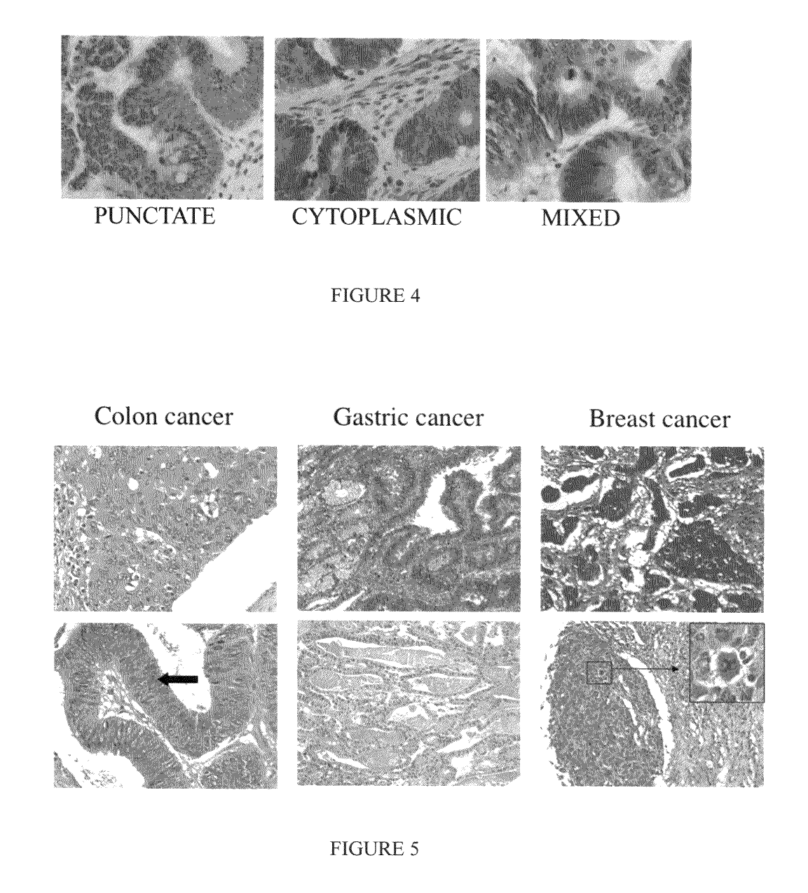

Using the method described herein it was observed that not only the level of methylation of arginine proteins was different in most cancerous cells, but the pattern of staining was also different. While no common pattern was observed across all normal tissues, the pattern was consistent for each tissue. Thus, the signal intensity of methylarginine proteins in normal endocrine tissues is highest in nuclei. The squamous epithelium of the normal skin exhibits methylation of arginine proteins in both cytoplasm and nucleus. In normal colon, in the epithelial cells, methylarginine proteins appear concentrated in a peri-nuclear location corresponding to the Golgi body. In the colon tumors examined, three patterns were observed as shown in FIG. 4. One pattern is labeled as punctuate and was similar to the normal pattern. The other two patterns, labeled as “cytoplasmic” or “mixed” were distinct from the normal pattern. Therefore, a finding of cytoplasmic or mixed pattern would be indicative of the presence of cancerous tissue, but a finding of punctuate pattern would need additional tests to confirm the presence or absence of cancerous tissue. Methylarginine expression is generally widely distributed throughout the tissues of the human body with several minor exceptions.

The present invention also provides kits for the detection of the level of or subcellular localization of methylation status of arginine containing proteins. The kits comprise an antibody (primary antibody) that specifically binds to arginine containing proteins when the arginine in the proteins is methylated, secondary antibody or other agents that can label the primary antibody, appropriate buffers etc.

The following specific examples are provided to illustrate the invention, but are not intended to be limiting in any way.

### Example 1

### Materials and Methods

### Tissues

Formalin-fixed paraffin-embedded human tissues were retrieved from the paraffin tissue archives of the Department of Pathology of the University of Verona. A minimum of two different samples for each normal tissue type was analyzed. Tissue samples from 136 individuals affected by pancreatic endocrine tumors were stained with methylarginine-specific antibody.

### Immunohistochemistry

Formalin fixed-paraffin-embedded tissues were cut into 4-micron thick sections and placed on poly-lysine coated slides. Antigen retrieval was performed in 10 mM citrate buffer, pH 6.0 heated in microwave at 360 W for 20 min. Endogenous peroxidase and non-specific sites were blocked by incubation, respectively, with 3% H₂O₂ and 10% goat serum for 15 min. at room temperature. When necessary, endogenous alkaline phosphatase was inhibited with 1 mM levamisole.

Sections were then incubated with the control antibody, or the methyl-specific antibody (3.75-5 μg/ml) for 1 h and 30 min. at room temperature, washed three times with PBST (PBS/0.2% Tween-20), and incubated for 30 min. at room temperature with the appropriate secondary antibodies. These included donkey anti-goat IgG biotin-conjugated (1:250) (Santa Cruz Biotechnology, Santa Cruz, Calif.) followed by streptavidin-AP conjugated (1:200) (Chemicon® International Temecula, Calif.); goat anti-mouse and anti-rabbit conjugated to either peroxidase-labelled dextran polymer (DakoEnVision+®, Peroxidase, Mouse and Rabbit Ready-to-use) or alkaline phosphatase (Dako EnVision®, Alkaline Phosphatase, Mouse and Rabbit Ready-to-use). After 3 washes with PBST, diaminobenzidine or fast red chromogen were respectively used as peroxidase or alkaline phosphatase substrates, for 10 min. incubations at room temperature.

### Results

The distribution pattern of the biomarker differs markedly in normal versus pathological biopsy material.

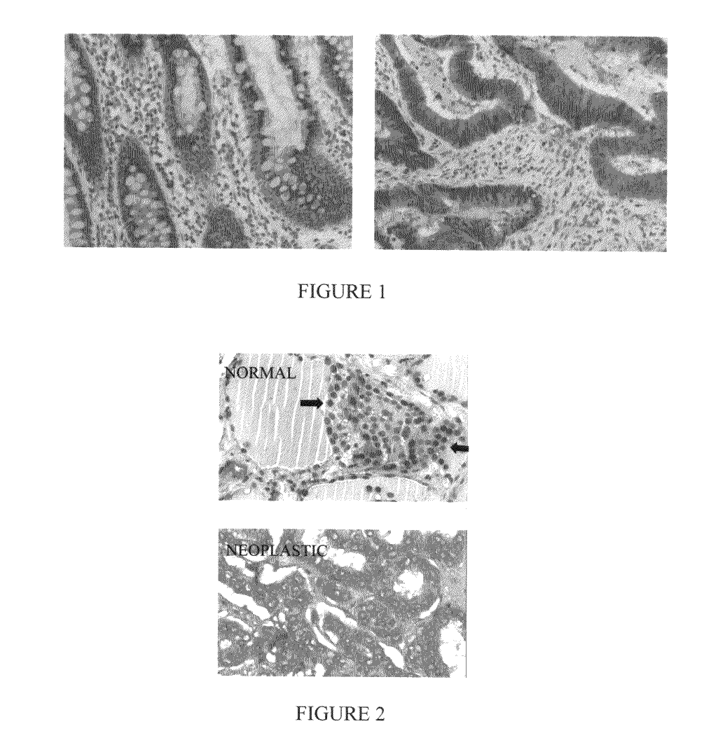

1. Colon Cancer: When the method is applied to compare normal colonic cells with a pathology series (108 cases) of cells from hyper-plastic polyps (HP), tubular adenomas (TA) and neoplastic cells from tumors biopsies of patients with colon cancer (CC), the normal colon cells demonstrate a punctate, perinuclear staining pattern (FIG. 1, left panel) that continued to be observed in the HP and some TA, with the beginning of diffuse cytoplasmic staining in the latter two. The CC specimens exhibit either the typical punctate/perinuclear stain or a diffuse cytoplasmic stain (FIG. 1, right panel) or a mixture of the two. The staining patterns observed in colon cancer specimens were statistically analyzed by the exact ratio likelihood chi-square test at a significance level of 0.05 (Table I). The p-value based on differentiation is <0.05. The p-values for stage, location and sex were not significant.

TABLE I
Colon Tumor Staining Pattern

| | Pattern | | | |
|---|---|---|---|---|
| Category | “Punctate” | Cytoplasmic | Mixed | |
| (Differentiation) | | | | |
| Well differentiated | 3 | 1 | 0 | n = 4 |
| Moderately differentiated | 12 | 23 | 9 | n = 44 |
| Poorly differentiated | 0 | 6 | 4 | n = 10 |
| (Stage) | | | | |
| T1 | 2 | 7 | 1 | n = 10 |
| T2 | 5 | 4 | 2 | n = 11 |
| T3 | 8 | 16 | 7 | n = 31 |
| T4 | 1 | 3 | 2 | n = 6 |
| (Location) | | | | |
| Right | 9 | 21 | 8 | n = 38 |
| Left | 6 | 9 | 5 | n = 20 |
| (Sex) | | | | |
| Male | 9 | 15 | 2 | n = 26 |
| Female | 6 | 16 | 10 | n = 32 |

The p values determined for the statistical significance of the differences between staining patterns and tumor characteristics are: 0.035 for tumor differentiation; 0.0575 for sex; 0.756 for tumor stage and 0.815 for tumor location.

2. Thyroid Cancer: When thyroid papillary carcinoma is compared with non-neoplastic thyroid tissue, marked differences of a different type are observed. Non-neoplastic normal thyroid presents a predominant nuclear stain arising from the methylarginine-specific antibody. Tumor tissue, on the other hand, shows methylarginine stain is mostly cytoplasmic.

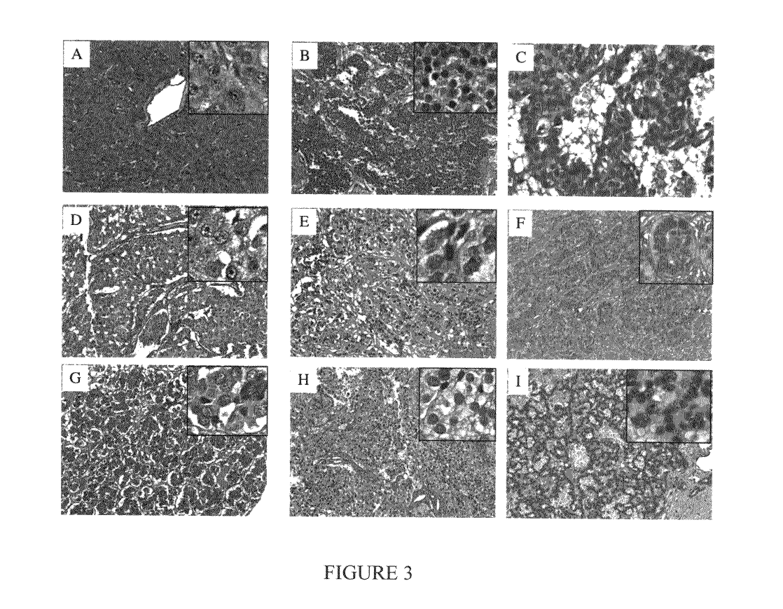

3. Pancreatic Cancer: In the largest series of 136 tumor specimens drawn from archival pancreatic endocrine tumors (PET), there are similar distinctive differences between normal and neoplastic segments of the biopsy material. Although normal endocrine pancreatic tissue uniformly presents a predominantly nuclear staining pattern, the staining pattern of PET tissue from human biopsies is diverse. As in the case of colon cancer, neoplastic tissue of PET can exhibit a range of staining patterns including cytoplasmic, mixed cytoplasm and nuclear, juxta-nuclear Golgi-like staining and weak to undetectable staining as well. FIG. 3 illustrates the extreme range of staining patterns that can be observed with PET.

The diversity of staining characteristics in this PET series suggests the existence of a heterogeneous patient population and, by implication, the possibility of differential patho-physiology involving methylarginine protein distribution. The method used to reveal these differences can be applied in future efforts to stratify cancer patient populations based on staining type with regard to the underlying molecular pathology and prospective therapeutic responses.

Thus the method of methylarginine biomarker detection provides innovation for the objective assessment of human tumors with regard to: (a) facilitation of disease diagnosis, (b) prognostic evaluation of disease status and progression and (c) stratification of human cancer types to promote the development of personalized medicine including a more informed selection of the most efficacious drug and/or therapeutic regimen for each patient.

Since normal biopsy specimens exhibit consistent patterns of tissue-specific methylarginine staining (i.e. distinctive punctate perinuclear pattern in colon, high signal intensity in nuclei and cytoplasm in endocrine pancreas and predominantly nuclear in thyroid), deviation from this appearance indicates pathology that involves protein methylation.

### Example 2

In normal tissue, immunohistochemical staining for methylarginine consistently stains the FFPE material in patterns characteristic of the tissue type. Endocrine tissue exhibits a predominantly nuclear stain and other tissues, in contrast, exhibit predominantly cytoplasmic staining. Tumor tissues differ from normal with respect to both the intensity of methylarginine staining and the sub-cellular location of the staining. Rather than exhibiting homogeneity of staining, as in normal tissues, neoplasms may yield negative reactions in as few as 5% of the cases (as in breast tumors) to as much as 20% to 30% of the cases observed in other tumor types (Table 2).

TABLE 2
Expression of methylarginine in various neoplasms

| | | | | % |
|---|---|---|---|---|
| Cancer type | Cases | positive | negative | negative |
| Thyroid papillary | 8 | 8 | 0 | 0 |
| Breast | 64 | 60 | 4 | 4.6 |
| Colon | 19 | 15 | 4 | 22 |
| Gastric | 32 | 25 | 7 | 21 |
| Pancreatic ductal | 25 | 17 | 8 | 32 |
| Pancreatic | 10 | 8 | 2 | 20 |
| endocrine | | | | |

In sub-categories of neoplasms, the sub-cellular localization of methylarginine stain also varies. In the small series of thyroid tumors, although all tumors yielded a positive staining reaction, the sub-cellular location varied from normal, staining predominantly cytoplasm rather than nucleus (FIG. 2). In the case of colon tumors (FIG. 4), three types of staining may be observed; staining may be “punctate” in a peri-nuclear golgi-like pattern which is typical of normal tissue, predominantly “cytoplasmic” or a “mixed” staining pattern showing elements of both staining types.

Another example of distinctive patterns of localization of methylarginine proteins is shown in FIG. 5 for colon, gastric and breast tissues.

While the invention has been described through specific embodiments, routine modifications will be apparent to those skilled in the art and such modifications are intended to be within the scope of the present invention.

What is claimed is:

1. A method for distinguishing a normal biological sample from a cancerous biological sample comprising the steps of comparing the level of binding or pattern of binding of antibodies specific to methylarginine proteins in the normal biological sample and a suspected cancerous biological sample, and identifying the suspected cancerous biological sample as cancerous if the level or pattern of binding is different from the level or pattern in the normal sample, wherein said antibodies specifically bind to peptides or proteins when one or more arginines is methylated, but does not specifically bind to peptides or proteins when the one or more arginines is not methylated.

2. The method of claim 1, wherein the antibody is an antibody which specifically binds to a peptide or protein comprising an epitope of at least 5 amino acids, and wherein said epitope has GRG motif.

3. The method of claim 1, wherein the normal biological sample and the suspected cancerous biological sample are obtained from the same individual.

4. The method of claim 1, wherein the biological samples are fixed and processed as paraffin embedded sections.

5. The method of claim 1, wherein the biological samples are processed as cryostat sections.

6. The method of claim 1, wherein the biological samples are obtained from a tissue selected from the group consisting of thyroid, breast, pancreas and colon.

7. The method of claim 6, wherein the pancreatic tissue is pancreatic ductal tissue or pancreatic endocrine tissue.

8. The method of claim 1, wherein the biological samples are obtained by biopsy.

9. The method of claim 1, wherein both the normal biological sample and the suspected cancerous biological sample are from the thyroid tissue, wherein the normal biological sample shows a predominantly subcellular binding of the antibodies to the nucleus and the cancerous biological sample shows a subcellular localization predominantly in the cytoplasm.

10. The method of claim 1, wherein both the normal biological sample and the suspected cancerous biological sample are from colon tissue, wherein the normal biological sample shows a predominantly subcellular localization of the methylated arginine containing proteins predominantly in the perinuclear region and the cancerous biological sample shows a “mixed” pattern of subcellular localization of the methylated arginine containing proteins in both the Golgi and cytoplasmic regions.

11. A method for identifying an abnormal status of methylation of arginine containing proteins in a test biological sample comprising the steps of:
a) obtaining a test biological sample;
b) comparing the level of binding or pattern of binding of antibodies specific to methylarginine proteins in the test biological sample to a normal control,
c) identifying the test biological sample as abnormal if the level or pattern of binding is different from the level or pattern of binding in the normal sample.

12. The method of claim 11, wherein the biological sample is obtained from thyroid, breast, pancreas, gastrointestinal tract or colon.

13. The method of claim 11, wherein the test and normal samples are obtained from the same individual.
